(19) United States
(12) Patent Application Publication
(10) Pub. No.: US 2023/0242841 A1
(43) Pub. Date: Aug. 3, 2023

(54) DEHYDRATED CLEANING COMPOSITIONS AND REUSABLE PACKAGING SYSTEMS INCLUDING THE SAME

(71) Applicant: Greenology Products, LLC, Raleigh, NC (US)

(72) Inventors: Adam McCarthy, Raleigh, NC (US); Rick Abbott, Raleigh, NC (US); Amy McGaughy, Raleigh, NC (US); Kayla Messier Jones, Wake Forest, NC (US)

(21) Appl. No.: 18/162,126

(22) Filed: Jan. 31, 2023

Related U.S. Application Data
(60) Provisional application No. 63/305,012, filed on Jan. 31, 2022.

(51) Int. Cl. C11D 1/04; C11D 3/10; C11D 1/14; C11D 7/50; C11D 17/00

(52) U.S. Cl. CPC C11D 1/04; C11D 3/10; C11D 1/143; C11D 7/5009; C11D 17/0047

(57) ABSTRACT

Compositions including an organic acid and a carbonate salt, wherein when the composition is subjected to a solvent, the composition effervesces and exhibits a sustained dissolution timeframe. The compositions may be in the form of a powder, tablet or gel and may also possess antimicrobial properties. Sustainable, reusable packaging systems including the compositions and products described herein are also provided.

### CROSS-REFERENCE TO RELATED APPLICATION

This application claims priority to U.S. Provisional Application No. 63/305,012, filed Jan. 31, 2022, the content of which is hereby incorporated herein by reference in its entirety.

### FIELD

The presently disclosed subject matter relates to compositions from which effervescent formulations can be prepared using a cold solvent. The compositions are prepared such that dissolution and effervescence commence within a desired time frame.

### BACKGROUND

Effervescent compositions are well known and improvements have been made in the quality of products that effervesce. However, issues such as speed of dissolution, degree of dissolution and sustained visual interest associated with dissolution continue to exist. Accordingly, there is a need for improved effervescing products and sustainable packaging to contain the same.

### SUMMARY

The presently disclosed subject matter provides compositions including an organic acid and a carbonate salt, wherein when the composition is subjected to a solvent, the composition effervesces and exhibits a sustained dissolution timeframe within a consumer window of interest of less than five (5) minutes, and in particular, about 30 to 180 seconds. In further embodiments, the composition is in the form of a powder, tablet or gel and may also possess antimicrobial properties.

Embodiments of the presently disclosed subject matter further provide products including the compositions described herein wherein the product is a cleaning composition, consumer product or personal care product.

In addition to cleaning properties, compositions embodied by the presently disclosed subject matter also display sanitizing and/or disinfecting properties.

According to further embodiments, the presently disclosed subject matter provides a reusable cleaner packaging system including a reusable container, a lid and a cleaning composition concentrate in the form of a powder, tablet or gel.

Embodiments of the presently disclosed subject matter further provide kits including the compositions, products and reusable cleaner packaging systems described herein and accompanying instructions for use thereof.

The compositions and methods described herein can be provided in the form of natural, organic or certified organic compositions and methods.

Certain aspects of the presently disclosed subject matter have been stated hereinabove. Other aspects will become evident as the description proceeds when taken in connection with the accompanying examples presented herein below.

### DETAILED DESCRIPTION

The presently disclosed subject matter now will be described more fully hereinafter with reference to the accompanying examples, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated examples. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises.” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items. Moreover, it should be noted that various embodiments of the presently disclosed compositions of matter may “comprise,” “consist” or “consist essentially of” the components or ingredients disclosed herein.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of any numerical ranges by endpoints or in succession includes all numbers, e.g., whole integers, including fractions thereof and decimals, subsumed within that range or sequence (for example, the recitation of 1 to 5 includes 1, 2, 3, 4, and 5, as well as fractions thereof, e.g., 1.5, 2.25, 3.75, 4.1, and the like and any range within that range and at least 95%, 96%, 97%, 98% or 99% includes all decimals in between and up to the next higher percentage up to 100).

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.

The presently disclosed subject matter provides compositions including an organic acid and a carbonate salt with improved speed to dissolution particularly in cold solvents. The compositions of this present inventive concept, once mixed into a solvent, dissolve to produce solutions capable of cleaning hard surfaces such as tile, glass, plastic, ceramic, granite, wood, stone or any hard surface typically found in a residential or commercial environment, as well as compositions that are suitable to clean humans and animals. In particular embodiments, the compositions described herein kill 99.999% of germs, are alcohol-free, bleach-free and/or phosphate-free.

According to particular embodiments, the compositions comprise, consists or consist essentially of an organic acid and a carbonate salt, wherein when the composition is subjected to a solvent the composition effervesces and dissolves over a period of less than about five (5) minutes, less than about three (3) minutes, and more specifically, in about 30 to 180 seconds. In some embodiments, the organic acid is at least one of citric acid, lactic acid, tartaric acid, malic acid or ascorbic acid. In some embodiments, the organic acid is present in an amount from about 5% to 60%, and in some embodiments, from about 8% to 55%.

In some embodiments, the carbonate salt is at least one of sodium sesquicarbonate, sodium bicarbonate, potassium carbonate or potassium bicarbonate. In particular embodiments, the carbonate salt is sodium carbonate. The selected carbonate salt provides high solubility in cold water contributing to the speed and mildness in which the reaction is able to happen and its sequestering character is an added benefit, once in solution. In some embodiments, the carbonate salt is present in an amount from about 5% to 30%, and in some embodiments, from about 8% to 26%.

In some embodiments, the temperature of the solvent is cold or warm. In particular embodiments, the solvent is water. In further embodiments, the water is in a temperature range from about 40 to 60° F. or in a temperature range from about 60 to 150° F. As used herein, “water” may refer to potable water, tap water, distilled water, demineralized water, de-ionized water, well water, recycled water, reclaimed water and non-potable water.

Compositions of the present inventive concept may also include at least one of a surfactant, chelant, flow agent, pH buffering agent, water softening agent, viscosifying agent, preservative, fragrance or moisturizer. In some embodiments, the surfactant is an anionic surfactant. The anionic surfactant may include one derived from carboxylic acids, sulfates, sulfonic acids, and phosphoric acid derivatives. In particular embodiments the anionic surfactant is sodium lauryl sulfoacetate or sodium olefin sulfonate.

In some embodiments, the surfactant is present in an amount from about 0% to 65%, and in some embodiments, from about 5% to 60%.

In some embodiments, the chelant is selected from gluconodeltalactone and sodium citrate. In some embodiments, the chelating agent is present in an amount from about 0% to 65%, and in some embodiments, from about 10% to 60%, and in some embodiments from about 15% to 60%.

In some embodiments, the viscosifying agent is selected from hydroxyethyl cellulose, hydroxypropyl methylcellulose and carboxymethylcellulose. The viscosifying or thickening agent is particularly beneficial for use in applications where cling and/or thicker flow characteristics are desirable, such as for a toilet bowl cleaner, or shower cleaner. Surprisingly, the dissolution action of the compositions described herein are sufficient to both disperse, hydrate and mix the ingredients, allowing the formulation to thicken uniformly, without clumping, in the presence of all ingredients. A gentle shake as the reaction is taking place ensures that the viscosity is uniformly attained. In some embodiments, the viscosifying agent is present in an amount from about 0% to 15%, from about 0% to 1%, from about 5% to 15%, and in some embodiments, from about 0.3% to 5%.

In some embodiments, the preservative selected from sodium benzoate, potassium sorbate and a plant extract preservative. The plant extract may be one as described in International Patent Application WO2020227422, which disclosure is incorporated herein by reference in its entirety. In some embodiments, the preservative is present in an amount from about 0% to 20%.

In some embodiments, the fragrance is an essential oil. Representative essential oils include, but are not limited to, allspice, ambrette seed, amyris, angelica root, anise, star anise, atlas cedarwood, Peru balsam, basil, holy basil, bay, bay laurel, beeswax absolute, benzoin absolute, bergamot, bergamot mint, black pepper, blue cypress, bois-de-rose, boronia, bursera graveolens, cajeput, cananga, cardamom, carrot seed, cassia, catnip/catmint, atlas cedarwood, Virginian cedarwood, German chamomile, Roman chamomile, chocolate peppermint, cinnamon, citronella, clary sage, clove bud, coffee, common sage, coriander, cumin, cypress, blue cypress, davana, dill, dalmation sage, elemi, eucalyptus, lemon eucalyptus, radiata eucalyptus, fennel, fir needle, frankincense, galbanum, geranium, rose geranium, ginger, grapefruit, gurjum balsam, helichrysum, hyssop, immortelle, jamine absolute, juniper berry, kanuka, abrialis lavandula, lavender, lavendin, lemon, lemon balm, lemon eucalyptus, lemongrass, lemon myrtle, lime, linden blossom, mandarin, mandravasarotra, manuka, marjoram, may chang, melissa, mint, mullein, myrrh, myrtle, lemon myrtle, neroli, niaouli, nutmeg, oakmoss, oliganum, bitter orange, sweet orange, oregano, palmarosa, palo santo, parsley, patchouli, peppermint, petitgrain, berry/leaf pimento, scotch pine, ravensara, rose, rosemary, rosewood, common sage, Spanish sage, sandalwood, saro, spearmint, spikenard, spruce, tagetes, tangerine, common tea tree, lemon tea tree, New Zealand tea tree, thyme, tobacco, tuberose, tulsi, vanilla, vetiver, violet leaf, yarrow, ylang ylang, and yuzu. In some embodiments, the fragrance is present in an amount from about 0% to 10%.

In some embodiments, the moisturizer is natural product. In some embodiments, the moisturizer is an oil derived from a natural product. Moisturizers suitable for the composition include, but are not limited to, algin (alginic acid), honey, coconut oil, olive oil, shea butter, olive oil, oat oil, avocado oil, sunflower oil, cucumber oil, almond oil, buttermilk, castor oil, rose water and mineral oil. In some embodiments, the moisturizer is present in an amount from about 0 to 10.6%.

Notably, compositions of the present inventive concept can exist in the form of a powder, tablet, capsule, gel or combination thereof. In particular embodiments, the composition is a powder. In some embodiments, the powder is a free-flowing powder with a density >1.0 g/ml. In particular embodiments, the powder surprisingly sinks in solution as opposed to rising and remaining on the top surface of the solvent, i.e., floating on top. In some embodiments, the composition is a single dose/use powder concentrate in a stick pack or loose powder in a canister. The composition can be readily converted to a spray product by adding a solvent such as water to a prescribed level to a spraying unit containing the powder.

Embodiments of the present inventive concept further provide compositions that possess mitigating properties. In such instances, the compositions prevent or significantly minimize contamination by pathogenic and non-pathogenic microorganisms, suppress microbial growth, reduce microbial infestation, treat products or surfaces to improve product resistance to microbial infestation, reduce biofilm, prevent conversion of bacteria to biofilm, prevent or inhibit microbial infection, prevent spoilage, retard or minimize or prevent quorum sensing, and/or retard microbial reproduction. In particular embodiments, the mitigation properties of the compositions of the present inventive concept allow the control of one or more specific microorganisms as described herein that are directly or indirectly infectious or pathogenic to humans (or both humans and animals).

The compositions may be used to reduce, inhibit or completely eliminate pathogen population in a variety of consumer products, such as personal care products; household, institutional and industrial (HI&1) care products; therapeutics; and others. The compositions described herein may be used to partially or completely replace currently available cleaning solutions, some of which are known as toxic to humans and animals, or at least reduce their concentration in such products for human or animal use. In particular embodiments, the compositions reduce the pathogen population and/or inhibit further microbial growth.

The pathogens against which the compositions may be effective include a wide spectrum of microorganisms such as bacteria, viruses, virinos, viroids, phages, fungi (including yeasts and molds) lichens, spores, protozoa and some algae, and further include combinations of the same type of microorganism as well as combinations of different types of microorganisms.

In some embodiments, the microorganism is a bacterium. In other embodiments, the bacterium may be a gram-positive bacterium. In still other embodiments, the bacterium may be a gram-negative bacterium. In particular embodiments, the bacterium is selected from Bordetella pertussis, Borrelia burgdorferi, Brucella abortus, Brucella canis, Brucella melitensis, Brucella suis, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli (E. coli), Enterotoxigenic Escherichia coli (ETEC), Enteropathogenic E. coli, Francisella tularensis, Haemophilus influenza, Helicobacter pylori, Klebsiella pneumoniae, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, methicillin resistant Staphylococcus aureus (MRSA), Staphylococcus epidermidis, Staphylococcus saprophyticus, Streptococcus agalactiae, Streptococcus mutans, Streptococcus pneumonia, Streptococcus pyogenes, Treponema pallidum, Vibrio cholera, Vibrio harveyi, Yersinia pestis and combinations thereof.

In some embodiments, the microorganism is a virus in particular embodiments, the virus is selected from Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus), rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus), human immunodeficiency virus type 1 (Retroviridae, Lentivirus); influenza A/B/C virus (Orthomyxoviridae, the common “flu” virus); a corona virus (Coronaviridae, SARS-CoV, HCoV NL63, HCoV HKU1, MERS-CoV and SARS-CoV-2); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); hepatitis delta virus (hepatitis D virus) (unassigned Family, Deltavirus); a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a norovirus, a parvovirus (e.g., parvovirus B19, canine parvovirus), or a picornavirus (e.g., poliovirus, hepatitis A), or combinations thereof.

In some embodiments, the microorganism is a fungus, selected in some embodiments from Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger, Blastomyces dermatitidis, Candida albicans, Candida albicans var. stellatoidea, Candida dublinensis, Candida glabrata, Candida guilliermondii, Candida krusei, Candida parapsilosis, Candida pelliculosa, Candida tropicalis, Cladophialophora carrionii, Coccidioides immitis, Cryptococcus neoformans, Cunninghamella sp., Epidermophyton floccosum, Exophiala dermatitidis, Filobasidiella neoformans, Fonsecaea pedrosoi, Geotrichum candidum, Histoplasma capsulatum, Hortaea werneckii, Issatschenkia orientalis, Madurella grisae, Malassezia furfur, Malassezia furfur complex, Malassezia globosa, Malassezia obtuse, Malassezia pachydermatis, Malassezia restricta, Malassezia slooffiae, Malassezia sympodialis, Microsporum canis, Microsporum fulvum, Microsporum gypseum, Microsporum gypseum complex, Microsporum gypseum, Mucor circinelloides, Nectria haematococca, Paecilomyces variotii, Paracoccidioides brasiliensis, Penicillium marneffei, Phialophora verrucosa, Pichia anomala, Pichia guilliermondii, Pneumocystis jirovecii, Pseudallescheria boydii, Rhizopus oryzae, Rodotorula rubra, Saccharomyces cerevisiae, Scedosporium apiospermum, Schizophyllum commune, Sporothrix schenckii, Stachybotrys chartarum, Trichophyton mentagrophytes, Trichophyton mentagrophytes complex, Trichophyton mentagrophytes, Trichophyton mentagrophytes, Trichophyton rubrum, Trichophyton tonsurans, Trichophyton verrucosum, Trichophyton violaceum, Trichosporon asahii, Trichosporon cutaneum, Trichosporon cutaneum complex, Trichosporon inkin and Trichosporon mucoides.

In some embodiments, the fungus is yeast, being selected, in some embodiments, from Candida albicans, Candida albicans var. stellatoidea, Candida dublinensis, Candida glabrata, Candida guilliermondii, Candida krusei, Candida parapsilosis, Candida pelliculosa, Candida tropicalis, Cryptococcus neoformans, Filobasidiella neoformans, Geotrichum candidum, Issatschenkia orientalis, Malassezia furfur, Malassezia pachydermatis, Pichia anomala, Pichia guilliermondii, Pneumocystis jirovecii, Rodotorula rubra, Trichosporon asahii, Trichosporon cutaneum, Trichosporon inkin and Trichosporon mucoides.

In some embodiments, the fungus is mold, being selected, in some embodiments, from Absidia corymbifera, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger, Cladophialophora carrionii, Coccidioides immitis, Epidermophyton floccosum, Exophiala dermatitidis, Fonsecaea pedrosoi, Hortaea werneckii, Madurella grisae, Microsporum canis, Microsporum fulvum, Microsporum gypseum, Microsporum gypseum, Microsporum gypseum, Mucor circinelloides, Nectria haematococca, Paecilomyces variotii, Paracoccidioides brasiliensis, Penicillium marnetfei, Pseudallescheria boydii, Rhizopus oryzae, Scedosporium apiospermum, Schizophyllum commune, Sporothrix schenckii, Stachybotrys chartarum, Trichophyton mentagrophytes complex. Trichophyton mentagrophytes, Trichophyton mentagrophytes, Trichophyton rubrum, Trichophyton tonsurans, Trichophyton verrucosum and Trichophyton violaceum.

In particular embodiments, the microorganisms are Escherichia coli, Staphylococcus aureus (including methicillin resistant Staphylococcus aureus), Pseudomonas aeruginosa, Klebsiella pneumoniae, Aspergillus brasiliensis and/or Candida albicans.

In particular embodiments, the microorganisms are non-pathogenic microorganisms. That is, the non-pathogenic microorganism does not typically cause disease and/or possesses attenuated pathogenicity.

The mitigating properties of the compositions of the present inventive concept render the compositions as suitable cleaners or cleaning additives. “Clean” or cleaning,” as used herein, refers to the ability to perform or aid in soil (e.g., oil, pigment, food, blood, protein, bleachable, etc.) removal, dirt removal, dust removal, stain removal, odor removal, microbial population reduction, or a combination thereof.

Further, compositions of the present inventive concept can serve as the active ingredient in a sanitizer. As used herein, “sanitizer” refers to a substance or composition that reduces, but not necessarily eliminates, microorganisms from the inanimate environment, including airborne microorganisms, to levels considered safe as determined by public health codes or regulations. Sanitizers include food contact and nonfood contact products as described herein.

Compositions of the present inventive concept can also serve as an active ingredient in a disinfectant. As used herein, “disinfectant” refers to a substance or composition that destroys, neutralizes or inhibits the growth of disease-carrying microorganisms. Further, a disinfectant may be used in the air, on hard, inanimate surfaces and/or objects to destroy or irreversibly inactivate fungi and bacteria but not necessarily their spores. In particular embodiments, the disinfectant exhibits general or broad-spectrum disinfectant efficacy. Accordingly, in some embodiments, the subject matter of the present invention is a broad-spectrum disinfectant having efficacy against both grain-negative and gram-positive bacteria. In some embodiments, the compositions of the present inventive concept may achieve total kill, at a log 6 reduction of organisms, at double the contact time required for sanitizing (i.e., 10 minutes versus 5 minutes). In further embodiments, the disinfectant compositions of the present inventive concept may include virucidal properties and/or bactericidal properties. In some embodiments, the disinfectant product may be used on hard, non-porous surfaces, in the air and in the form of products as further described below. In still other embodiments, the disinfectant may be a high-level disinfectant, an intermediate-level disinfectant or a low-level disinfectant. “High-level disinfection” or “high-level disinfectant” refers to a compound or composition that kills substantially all organisms, except high levels of bacterial spores, and is effected with a chemical germicide cleared for marketing as a sterilant by the U.S. Food and Drug Administration. As used herein, the term “intermediate-level disinfection” or “intermediate-level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low-level disinfectant” refers to a compound or composition that kills some viruses and bacteria with a chemical germicide registered as a hospital disinfectant by the EPA.

In some embodiments, compositions of the present inventive concept can clean and sanitize and/or disinfect in one step.

As discussed above, in some embodiments, the product is a cleaning composition. In particular, the cleaning composition is a product for human or animal care and/or use. In further embodiments, the cleaning composition is a laundry detergent, laundry additive, fabric sanitizer, automatic or manual dishwashing detergent, multi-surface cleaner, rinse-free antibacterial hard surface soap, hard surface cleaner, hand soap, rinse-free hand soap, hand sanitizer, body soap, body wash, deodorizing spray, shampoo or animal care cleaning products such as shampoo, cage cleaner, rinse-free antibacterial hard surface soap, deodorizing spray, etc. In particular embodiments, the cleaning compositions are sulfate free, natural, organic and/or certified organic cleaning compositions including laundry detergents, stain removers, laundry pre-wash formulations, fabric sanitizers automatic or manual dishwashing detergents, multi-surface cleaners, hard surface cleaners, rinse-free antibacterial hard surface soaps, hand soaps, rinse-free hand soap, hand sanitizers, body soap, body wash, shampoo or animal cleaning products such as shampoo, cage cleaner, cleaning wipes, deodorizing spray, etc. See U.S. Pat. Nos. 9,447,366; 9,217,127; 9,217,125 and 8,835.370.

The presently disclosed subject matter provides compositions for use as a mitigating agent and/or composition generally. The compositions described herein can be provided in the form of a natural, organic or certified organic composition. As such, the compositions can provide effective mitigating activity, are generally regarded as safe (or safer) to use and generally exhibit no greater toxicity (or are less toxic) to the user and the environment than existing preservative and/or mitigating compositions used in the same capacity while also complying with the requirements of the United States Department of Agriculture's (USDA's) National Organic Program (NOP) for products labeled as “organic” or “made with organic.”

As used herein, the term “natural” refers to the state of being obtained from a natural source (plant or non-plant source, e.g., animal or insect source) in a substantially unmodified form, namely in the form which exists in the material natural origin, or a composition including components that are obtained in the aforementioned manner. Natural components may be obtained from their natural source by way of extraction or other ways of separation. “Natural” is also intended to encompass green, sustainable, holistic, bio-degradable, nature-identical, homeopathic, eco-friendly, earth-friendly, environmentally safe, paraben-free and/or non-toxic.

As used herein, the term “organic” refers to a product or ingredient that complies with 7 CFR § 205.301, which establishes organic content requirements for labeling and marketing purposes. Four categories of organic content have been established by the United States Department of Agriculture (USDA) under the National Organic Program (NOP): “100 percent organic,” which is defined as products comprising 100 percent organic ingredients; “organic,” which is defined as products comprising 95 percent or more organic ingredients; “made with organic,” which is defined as products comprising between 70 percent and 95 percent organic ingredients; and “ingredient panel only,” which is defined as products comprising less than 70 percent organic ingredients.

More particularly, under 7 CFR § 205.301, all of the ingredients in products sold, labeled, or represented as “100 percent organic” must be certified organic and contain (by weight or fluid volume) 100 percent organically produced ingredients.

Products sold, labeled, or represented as “organic” must contain (by weight or fluid volume) not less than 95 percent organically produced raw or processed agricultural products. Any remaining product ingredients must be organically produced, unless not commercially available in organic form, or must be nonagricultural substances or non-organically produced agricultural products as specified on the National List under 7 CFR § 205.605. Accordingly, non-organic ingredients allowed per National List may be used, up to a combined total of five percent of non-organic content.

Products sold, labeled, or represented as “made with organic” must contain (by weight or fluid volume) at least 70 percent organically produced ingredients. Accordingly, multi-ingredient agricultural products in the “made with organic” category must include at least 70 percent certified organic ingredients Although any remaining agricultural products are not required to be organically produced, non-agricultural products must be specifically allowed on the National List under 7 CFR § 205.605.

Multi-ingredient products having less than 70 percent certified organic content are not qualified to be certified. Accordingly, any non-certified product must not include the USDA organic seal anywhere or the word “organic” on a principal display panel. Such products may only list certified organic ingredients as organic in the ingredient list and the percentage of organic ingredients. Remaining ingredients are not required to follow the USDA organic regulations.

Further, under 7 CFR § 205.605, specified nonagricultural (nonorganic) substances may be used as ingredients in or on processed products labeled as “organic” or “made with organic,” including the following nonsynthetic and synthetic ingredients:

Nonsynthetics: acids (alginic; citric—produced by microbial fermentation of carbohydrate substances; and lactic), agar-agar; animal enzymes—(rennet—animals derived, catalase—bovine liver; animal lipase; pancreatin; pepsin; and trypsin); bentonite; calcium carbonate; calcium chloride; calcium sulfate-mined; carrageenan; dairy cultures; diatomaceous earth-food filtering aid only, egg white lysozyme (CAS #9001-63-2); enzymes derived from edible, nontoxic plants, nonpathogenic fungi, or nonpathogenic bacteria; flavors, nonsynthetic sources only and must not be produced using synthetic solvents and carrier systems or any artificial preservative; gellan gum (CAS #71010-52-1)—high-acyl form only; glucono delta-lactone-production by the oxidation of D-glucose with bromine water is prohibited; kaolin; L-malic acid (CAS #97-67-6); magnesium sulfate, nonsynthetic sources only; microorganisms-any food grade bacteria, fungi, and other microorganism; nitrogen-oil-free grades; oxygen-oil-free grades; perlite—for use only as a filter aid in food processing; potassium chloride; potassium iodide; sodium bicarbonate; sodium carbonate; tartaric acid-made from grape wine; waxes-nonsynthetic (carnauba wax; and wood resin); yeast-nonsynthetic, growth on petrochemical substrate and sulfite waste liquor is prohibited (autolysate; bakers; brewers; nutritional; and smoked-nonsynthetic smoke flavoring process must be documented); and synthetics: activated charcoal (CAS #s 744044-0; 64365-11-3)—only from vegetative sources; for use only as a filtering aid; alginates; ammonium bicarbonate—for use only as a leavening agent; ammonium carbonate—for use only as a leavening agent; ascorbic acid; calcium citrate; calcium hydroxide; calcium phosphates (monobasic, dibasic, and tribasic); carbon dioxide; cellulose—for use in regenerative casings, as an anti-caking agent (non-chlorine bleached) and filtering aid; chlorine materials-disinfecting and sanitizing food contact surfaces, except that residual chlorine levels in the water shall not exceed the maximum residual disinfectant limit under the Safe Drinking Water Act (calcium hypochlorite; chlorine dioxide; and sodium hypochlorite); cyclohexylamine (CAS #108-91-8)—for use only as a boiler water additive for packaging sterilization, diethylaminoethanol (CAS #100-37-8)—for use only as a boiler water additive for packaging sterilization, ethylene-allowed for postharvest ripening of tropical fruit and degreening of citrus: ferrous sulfate—for iron enrichment or fortification of foods when required by regulation or recommended (independent organization); glycerides (mono and di)—for use only in drum drying of food; glycerin-produced by hydrolysis of fats and oils; hydrogen peroxide, lecithin-bleached, magnesium carbonate—for use only in agricultural products labeled “made with organic” prohibited in agricultural products labeled “organic”, magnesium chloride-derived from sea water; magnesium stearate—for use only in agricultural products labeled “made with organic” prohibited in agricultural products labeled “organic”; nutrient vitamins and minerals, in accordance with 21 CFR 104; 20, Nutritional Quality Guidelines For Foods; octadecylamine (CAS #124-30-1)—for use only as a boiler water additive for packaging sterilization; ozone; pectin (low-methoxy); peracetic acid/peroxyacetic acid (CAS #79-21-0)—for use in wash and/or rinse water according to FDA limitations: for use as a sanitizer on food contact surfaces; phosphoric acid-cleaning of food-contact surfaces and equipment only; potassium acid tartrate, potassium carbonate; potassium citrate; potassium hydroxide-prohibited for use in lye peeling of fruits and vegetables except when used for peeling peaches during the individually Quick Frozen (IQF) production process; potassium iodide——for use only in agricultural products labeled “made with organic,” prohibited in agricultural products labeled “organic”; potassium phosphate—for use only in agricultural products labeled “made with organic,” prohibited in agricultural products labeled “organic”; silicon dioxide; sodium acid pyrophosphate (CAS #7758-16-9)—for use only as a leavening agent; sodium citrate; sodium hydroxide-prohibited for use in lye peeling of fruits and vegetables; sodium phosphates—for use only in dairy foods; sulfur dioxide—for use only in wine labeled “made with organic grapes,” provided that total sulfite concentration does not exceed 100 ppm; tartaric acid-made from malic acid; tetrasodium pyrophosphate (CAS #7722-88-5)—for use only in meat analog products, tocopherols-derived from vegetable oil when rosemary extracts are not a suitable alternative; and xanthan gum.

Further, the natural, organic and/or certified organic compositions described herein are substantially free of parabens, and in particular embodiments, paraben free. Parabens are man-made synthetic esters of p-hydroxybenzoic acid and have been used as a preservative in personal care, pharmaceutical and food products Exemplary parabens include, but are not limited to, methylparaben, ethylparaben, propylparaben, butylparaben, heptylparaben, isobutylparaben, isopropylparaben, benzylparaben and their sodium salts. As used herein, “substantially free” generally refers to a level considered to be a trace amount or background level. Such levels do not cause material harm that may be associated with parabens. Further, substantially free also indicates that parabens have not been intentionally added to the compositions, plant infusions or extracts or methods described herein. In particular embodiments, substantially free refers to the presence of parabens at a concentration of about 0.010% or less or optionally about 0.001% or less. In particular embodiments of the present inventive concept, the natural, organic and/or certified organic compositions described herein do not contain any parabens.

Embodiments of the present inventive concept further provide methods for making the compositions described herein. In general, compositions described herein can be manufactured using a batch process suitable for powder blending. Ingredients may be added sequentially, one at a time, into a suitable powder blender and mixed until homogenous and fully blended or some ingredients may be added simultaneously also providing a homogenous and fully blended product. As understood by one of ordinary skill in the art, a ribbon blender, screw blender or similar equipment may be used to blend powders. Purification steps are optional, and in most cases, purification is not necessary. Quality control procedures to assess pH, color, appearance, consistency, performance, etc. may be conducted.

Embodiments of the present inventive concept further provide a reusable packaging system. The reusable packaging system includes a container, a lid and a concentrated cleaning composition. In some embodiments, the container is composed of a sustainable material. As used herein, a sustainable material refers to materials produced in required volumes without depleting non-renewable resources and without disrupting the established steady-state equilibrium of the environment and key natural resource systems. Sustainable materials include those such as recyclable materials and/or those made entirely from natural materials.

In some embodiments, the container is composed of a recyclable material. In some embodiments, the container is composed of post-consumer recycled material, such as post-consumer recycled plastic, glass, metal or paper. In some embodiments, the container is a glass, plastic or metal container or combinations thereof. A metal container may include aluminum, steel, magnesium, iron, manganese and combinations thereof. Notably, the container can be cleaned and refilled infinitely thereby significantly reducing packaging disposal. In some embodiments, the container is refillable and/or recyclable. In particular embodiments, the sustainable and reusable packaging system allows for up to a 100% reduction in shipping water and up to a 100% reduction in plastic bottle pollution. In some embodiments, the cleaning composition concentrate is a composition described herein in the form of a powder, tablet, capsule, gel or combination thereof. In some embodiments, the metal container is an aluminum container. In some embodiments, the container is a glass or glass-like (i.e., glass-alternative) container. In particular embodiments, the container is not a conventional disposable container. That is, the container is intended to be reusable and refillable over multiple uses with the compositions according to the present inventive concept. In some embodiments the lid is a push-pull lid, a trigger spray lid, a spray cap lid or a spout lid. In some embodiments, the reusable packaging system is an all-purpose cleaner packaging system. In some embodiments, the all-purpose cleaner is a household cleaner. In some embodiments, the reusable packaging system results in usage of at least 50%, 60%, 70%, 80% or 90% less packaging than conventional disposable packaging systems used to package cleaning compositions.

The present inventive concept further provides kits or an assembly of components packaged together with optional instructions (written, audio or visual) regarding how to use the components of the kit. In particular embodiments, kits including components such as compositions, products and reusable packaging systems described herein accompanied by instructions for use thereof are provided. In particular embodiments, all packaging used to contain the components of the kit are composed of a reusable, recyclable and/or a post-consumer recycled material, such as post-consumer recycled plastic, glass, metal or paper.

The general procedures for making and using the compositions and/or products including the same of the present invention can be readily understood and appreciated by one skilled in the art. Some aspects of the present invention are described in more detail in the following non-limiting examples. The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1

### Preparation of Effervescent Composition

By way of example, approximately 6.0 g powder is dispensed into a bottle. The bottle is filled with 16-28 fl oz of tap water (tap water is typically at a temperature of 50-65° F.). Shake and wait approximately 1 minute, until fully dissolved; insert trigger sprayer or foaming pump and product is ready to use. The level of viscosity is less than or similar to conventional liquid dish detergents or liquid toilet bowl cleaners Notably, viscosity is controlled so as to reduce or eliminate clogging of the trigger sprayer, foaming pump, etc. and/or to minimize interference with fast dissolution.

Testing methods for the compositions described herein included those that are accepted industry standards known to those skilled in the art to evaluate performance testing, claims substantiation, etc.

### Exemplary Effervescent Composition

| Chemical Type | % Formula 1 | % Formula 2 | % Formula 3 |
|---|---|---|---|
| Anionic Surfactants | 1-40% | 5-31% | 5-58% |
| (e.g., Sodium Lauryl | | | |
| Sulfoacetate/Sodium | | | |
| Olefin Sulfonate) | | | |
| Chelating/Sequestering | 10-60% | 16-53% | 10.7-47.6% |
| Agents (e.g., GDL, | | | |
| Trisodium Citrate, | | | |
| Sodium Sesquicarbonate) | | | |
| Flow Agent (e.g., Silica) | 0.0-0.5% | 0.3-0.5% | 0.3-1.0% |
| Preservative (e.g., Sodium | 0-20% | 10-20% | 10-20% |
| Benzoate) | | | |
| Organic Acid/Acidifer/ | 1-55% | 5-55% | 5.5-54% |
| Cleaning Agent | | | |
| Fragrance | 0-10% | 1.5-10% | 1.75%-8.5% |
| Moisturizer (e.g., Algin) | 0-10% | 5-10% | 0-10.6% |
| Thickener (e.g., HEC) | 5-15% | 5-15% | |

### Example 2

| Ingredient | % |
|---|---|
| Glass Cleaner Powder (1) | |
| Sodium Lauryl Sulfoacetate | 5.00 |
| Hydrated Silica | 0.40 |
| Citric Acid | 86.60 |
| Sodium Sesquicarbonate | 2.00 |
| Trisodium Citrate | 3.00 |
| Fragrance | 3.00 |
| Total | 100.00 |
| Glass Cleaner Powder (2) | |
| Sodium Lauryl Sulfoacetate | 5.00 |
| Hydrated silica | 0.40 |
| Citric Acid | 19.00 |
| Glucono delta-lactone (GDL) | 19.00 |
| Sodium sesquicarbonate | 13.00 |
| Anhydrous Trisodium Citrate | 14.85 |
| sodium benzoate | 27.00 |
| Fragrance | 1.75 |
| Total | 100.00 |
| Glass Cleaner Powder (3) | |
| Sodium Lauryl Sulfoacetate | 5.00 |
| Hydrated silica | 0.40 |
| Citric Acid | 19.00 |
| GDL | 25.00 |
| Sodium sesquicarbonate | 13.00 |
| Anhydrous Trisodium Citrate | 15.00 |
| sodium benzoate | 20.00 |
| Fragrance | 2.6 |
| Total | 100.00 |

| Ingredient | % |
|---|---|
| Multi-surface Cleaner Powder (1) | |
| Sodium Lauryl Sulfoacetate | 20.00 |
| Sodium C14-16 Olefin Sulfonate | 4.00 |
| Hydrated Silica | 0.40 |
| Citric Acid | 18.60 |
| GDL | 19.00 |
| Sodium Sesquicarbonate | 2.00 |
| Trisodium Citrate | 13.00 |
| Sodium Benzoate | 20.00 |
| Fragrance | 3.00 |
| Total | 100.00 |
| Multi-surface Cleaner Powder (2) | |
| Sodium Lauryl Sulfoacetate | 12.00 |
| Hydrated silica | 0.60 |
| Citric Acid | 19.00 |
| GDL | 19.00 |
| Sodium sesquicarbonate | 8.00 |
| Anhydrous Trisodium Citrate | 12.65 |
| sodium benzoate | 27.00 |
| Fragrance | 1.75 |
| Total | 100.00 |

| Ingredient | % |
|---|---|
| Tub & Tile Cleaner Powder (1) | |
| Sodium Lauryl Sulfoacetate | 10.00 |
| Sodium C14-16 Olefin Sulfonate | 6.00 |
| Hydrated Silica | 0.40 |
| Citric Acid | 55.00 |
| GDL | 8.00 |
| Sodium Sesquicarbonate | 11.00 |
| Trisodium Citrate | 6.60 |
| Fragrance | 3.00 |
| Total | 100.00 |
| Tub & Tile Cleaner Powder (2) | |
| Sodium Lauryl Sulfoacetate | 10.00 |
| Sodium C14-16 Olefin Sulfonate | 6.00 |
| Hydrated Silica | 0.40 |
| Citric Acid | 54.00 |
| GDL | 8.00 |
| Sodium Sesquicarbonate | 2.00 |
| Trisodium Citrate | 6.60 |
| Sodium Benzoate | 10.00 |
| Fragrance | 3.00 |
| Total | 100.00 |
| Tub & Tile Cleaner Powder (3) | |
| Sodium Lauryl Sulfoacetate | 12.00 |
| Hydrated silica | 0.50 |
| Citric Acid | 41.00 |
| GDL | 35.00 |
| Sodium sesquicarbonate | 2.00 |
| Anhydrous Trisodium Citrate | 2.75 |
| sodium citrate | 5.00 |
| Fragrance | 1.75 |
| Total | 100.00 |

| Multi-Surface Floor Cleaner Powder | |
|---|---|
| Ingredient | % |
| Sodium Lauryl Sulfoacetate | 10.00 |
| Hydrated Silica | 0.40 |
| Citric Acid | 21.00 |
| GDL | 21.85 |
| Sodium Benzoate | 18.00 |
| Trisodium Citrate | 25.75 |
| Fragrance | 3.00 |
| Total | 100.00 |

| Ingredient | % |
|---|---|
| Sanitizer Cleaner Powder (1) | |
| Sodium C14-16 Olefin Sulfonate | 12.00 |
| Hydrated Silica | 0.50 |
| Citric Acid | 46.75 |
| GDL | 31.25 |
| Sodium Sesquicarbonate | 2.00 |
| Trisodium Citrate | 2.75 |
| Trisodium Citrate Dihydrate | 3.00 |
| Fragrance | 3.00 |
| Total | 100.00 |
| Sanitizer Cleaner Powder (2) | |
| Sodium C14-16 Olefin Sulfonate | 12.00 |
| Hydrated Silica | 0.50 |
| Citric Acid | 47.12 |
| GDL | 30.88 |
| Sodium Sesquicarbonate | 2.00 |
| Trisodium Citrate | 2.75 |
| Trisodium Citrate Dihydrate | 3.00 |
| Fragrance | 1.75 |
| Total | 100.00 |
| Sanitizer Cleaner Powder (3) | |
| Sodium C14-16 Alpha Olefin Sulfonate | 23.00 |
| Hydrated Silica | 0.50 |
| Citric Acid | 20.00 |
| Sodium Lauryl Sulfoacetate | 15.25 |
| Hydroxyethylcellulose | 25.00 |
| Sodium sesquicarbonate | 2.00 |
| sodium benzoate | 12.50 |
| Fragrance | 1.75 |
| Total | 100.00 |

| Foaming Dish Soap | |
|---|---|
| Ingredient | % |
| Sodium Lauryl Sulfoacetate | 50.00 |
| Sodium C14-16 Olefin Sulfonate | 8.00 |
| Hydrated Silica | 0.30 |
| Citric Acid | 8.00 |
| Sodium Sesquicarbonate | 2.00 |
| Trisodium Citrate | 8.70 |
| Sodium Benzoate | 20.00 |
| Fragrance | 3.00 |
| Total | 100.00 |

| Foaming Hand Soap | |
|---|---|
| Ingredient | % |
| Sodium Lauryl Sulfoacetate | 8.50 |
| Sodium C14-16 Olefin Sulfonate | 5.00 |
| Hydrated Silica | 1.00 |
| Citric Acid | 5.50 |
| GDL | 18.90 |
| Sodium Sesquicarbonate | 2.00 |
| Trisodium Citrate | 20.00 |
| Algin | 10.60 |
| Sodium Benzoate | 20.00 |
| Fragrance | 8.50 |
| Total | 100.00 |

### Example 3

Multi-Surface Floor Cleaner (ASTM D4488-A5 Test Method)

| | % Cleaning | Standard | |
|---|---|---|---|
| Sample | Efficiency | Deviation | P value |
| Multi-Surface Floor Cleaner | 99.72 | 1.55 | |
| Powder | | | |
| Competitive Product 1 | 97.93 | 5.16 | 0.5956 |
| Competitive Product 2 | 94.21 | 6.08 | 0.2030 |
| Competitive Product 3 | 99.43 | 0.75 | 0.7898 |

Results—No significant differences between average results

### Example 4

All Purpose Cleaner (ASTM D4488-A5 Test Method)

| | % Cleaning | Standard | |
|---|---|---|---|
| Sample | Efficiency | Deviation | P value |
| All Purpose Cleaner Powder | 94.89 | 0.74 | |
| Competitive Product 1 | 95.90 | 0.51 | 0.1229 |
| Competitive Product 2 | 95.96 | 1.81 | 0.3946 |

Results—No significant differences between average results

### Example 5

Tub & Tile Cleaner (HCPA DCC-16 Test Method)

| | % Cleaning | Standard | |
|---|---|---|---|
| Sample | Efficiency | Deviation | P value |
| Tub & Tile Cleaner Powder | 48.98 | 7.38 | |
| Competitive Product 1 | 40.67 | 5.92 | 0.0263 |

Results—Tub & Tile Cleaner performed significantly better than the competitive product

### Example 6

Glass Cleaner (HCPA DCC-09 Test Method)

| | | Standard | |
|---|---|---|---|
| Sample | Average Cleaning | Deviation | P value |
| Glass Cleaner Powder | 3.7 | 1.76 | |
| Competitive Product 1 | 3.2 | 1.1 | 0.20 |
| Competitive Product 2 | 3.1 | 1.8 | 0.21 |

Results—No significant difference between average results

### Example 7

Hand Dish Soap (HCPA DCC-18 Modified)

| | Hand Dish Soap Powder | Competitive Product |
|---|---|---|
| Tile # | % Soil Removal | % Soil Removal |
| Tile 1 | 100% | 97.9% |
| Tile 2 | 97.9% | 93.6% |
| Tile 3 | 95.7% | 83.0% |
| Tile 4 | 89.4% | 78.7% |
| Tile 5 | 87.2% | 74.5% |

Results—Hand Dish Soap performed significantly better than the competitive product

### Example 8

### Sanitizer Cleaner Powder (ASTM E1153 Non-Food Contact Sanitizer)

Test microorganism: Klebsiella aerogenes ATCC 13048
Efficacy Result: Following a 5 minute contact time, the geometric mean of Control carreirs was 1.48×10\hat{ }8. Lot 22-23 had a 99.994% reduction. Lot 22-24 had a 99.9995% reduction. Lot 22-25 had a 99.9997% reduction.
Test microorganism: Staphylococcus aureus ATCC 6538
Efficacy Result: Following a 5 minute contact time, Geometric mean of the Control counts was 1.06×10\hat{m}7. Lot 22-23 had a ≥99.9998% reduction. Lot 22-24 had a ≥99.99991% reduction. Lot 22-25 had a ≥99.9998% reduction
Results—Sanitizer Cleaner met the EPA requirements of a minimum log reduction of 99.9% for registration as a hard surface cleaner.

### Example 9

### One-Time Use All-Purpose Cleaner Packaging vs. Reusable Household Cleaner Packaging and Refill Concentrate

This comparison reviews the differences in packaging used to deliver a conventional all-purpose cleaner (28 fluid ounces) versus the reusable packaging system of the present inventive concept (Test), which in this example, includes a reusable aluminum bottle (16 fluid ounces), a trigger spray and powdered concentrate refills. The resulting system produces significantly less packaging usage than standard disposable bottles with spray caps.

Packaging information was developed for seven purchases. For one-time use, this included seven bottles and trigger caps. For reusable packaging, it included one bottle and trigger cap along with a concentrate sleeve; followed by 6 concentrated sleeves, packed two to a paperboard box (3 boxes in all).

Because the two products come in different size bottles, the data was normalized to create equivalency. Normalization was based on comparing the materials needed to deliver 3.5 gallons of cleaner, which is 16 one-time use containers and 28 reusable packaging containers.

Final data was provided in both grams and pounds of packaging needed to deliver 3.5 gallons of product.

| | Per | 1^{st} | Purchases | Purchases | Purchases | Purchases | Normalized @ | |
|---|---|---|---|---|---|---|---|---|
| | Container | Purchase | 2 + 3 | 4 + 5 | 6 + 7 | 1-7 | 3.5 Gallons | |
| | (Grams) | (Grams) | (Grams) | (Grams) | (Grams) | (Grams) | (Grams) | (LBs.) |
| Method 28 Fl. Oz. | | | | | | | | |
| PET Plastic Bottle | 43.6 | 43.6 | 87.2 | 87.2 | 87.2 | 305.2 | 597.6 | 1.5 |
| Plastic Label | 0.2 | 0.2 | 0.4 | 0.4 | 0.4 | 1.4 | 3.2 | 0.0 |
| Sub-Total | 43.8 | 43.8 | 87.6 | 87.6 | 87.6 | 306.6 | 700.8 | 1.5 |
| Trigger Spray Cap & Tube | 21.5 | 21.5 | 43.0 | 43.0 | 43.0 | 150.5 | 344.0 | 0.8 |
| TOTAL Method | 65.3 | 65.3 | 130.6 | 130.6 | 130.6 | 457.1 | 1044.8 | 2.3 |
| Test 16 Fl. Oz. | | | | | | | | |
| Aluminum Bottle | 60.8 | 60.8 | 0.00 | 0.0 | 0.0 | 60.8 | 60.8 | 0.1 |
| Trigger Spray Cap & Tube | 30.9 | 30.9 | 0.00 | 0.0 | 0.0 | 30.9 | 30.9 | 0.1 |
| Sub-Total | 91.7 | 91.7 | 0.00 | 0.0 | 0.0 | 91.7 | 91.7 | 0.2 |
| Plastic Concentrate Packet | 1.0 | 1.0 | 0.00 | 0.0 | 0.0 | 1.0 | 1.0 | 0.0 |
| TOTAL | 92.7 | 92.7 | 0.00 | 0.0 | 0.0 | 92.7 | 92.7 | 0.2 |
| Refills | | | | | | | | |
| Paperband Box | 5.8 | 0.0 | 5.8 | 5.8 | 5.8 | 17.4 | 78.3 | 0.2 |
| 2 Plastic Packets | 2.0 | 0.0 | 2.0 | 2.0 | 2.0 | 6.0 | 27.0 | 0.1 |
| Sub-Total | 7.8 | 0.0 | 7.8 | 7.8 | 7.8 | 23.4 | 105.3 | 0.2 |
| TOTAL Test | | 92.7 | 7.8 | 7.8 | 7.8 | 116.0 | 198.0 | 0.4 |

### Results

To deliver the same amount of household cleaner (3.5 gallons), the packaging system of the present inventive concept utilized 81% less packaging than the Method packaging system −0.4 pounds vs. 2.3 pounds.

The unique combination of an organic acid and carbonate salt will effervesce once mixed with water to improve dissolution. This effervescing action, along with the density of the compositional ingredients, allows for improved dissolution through gentle shaking/inversion without the need of high shear mixing. The concentrated form of the compositions described herein provide cleaning, disinfectant and/or sanitizing capabilities while eliminating the need to transport water over great distances and also minimizes the amount of packaging required where the proposed system of reusable bottles and concentrated refills uses significantly less packaging than the conventional approach of full-strength products sold in disposable containers. Such aspects of the present inventive concept benefit both human and animal health and the environment by making cleaning, sanitizing and disinfecting products and its associated packaging accessible, transportable, effective and reusable.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising an organic acid and a carbonate salt, wherein when the composition is subjected to a solvent, the composition effervesces and dissolves over a period of less than about five (5) minutes.

2. The composition of claim 1, wherein the temperature of the solvent is cold or warm.

3. The composition of claim 1, wherein the solvent is water.

4. The composition of claim 1, wherein the organic acid is at least one of citric acid, lactic acid, tartaric acid, malic acid or ascorbic acid.

5. The composition of claim 1, wherein the carbonate salt is at least one of sodium sesquicarbonate, sodium bicarbonate, potassium carbonate or potassium bicarbonate.

6. The composition of claim 1, wherein the composition further comprises at least one of a surfactant, chelant, flow agent, viscosifying agent, preservative, fragrance or moisturizer.

7. The composition of claim 6, wherein the composition comprises at least one anionic surfactant selected from the group consisting of sodium lauryl sulfoacetate and sodium olefin sulfonate.

8. The composition of claim 6, wherein the composition comprises at least one chelant selected from the group consisting of gluconodeltalactone and sodium citrate.

9. The composition of claim 6, wherein the composition comprises at least one flow agent comprising silica.

10. The composition of claim 6, wherein the composition comprises at least one viscosifying agent selected from the group consisting of hydroxyethyl cellulose, hydroxypropyl methylcellulose and carboxymethylcellulose.

11. The composition of claim 6, wherein the composition comprises at least one preservative selected from the group consisting of sodium benzoate, potassium sorbate and a plant extract preservative.

12. The composition of claim 6, wherein the composition comprises at least one fragrance comprising an essential oil.

13. The composition of claim 1, wherein the composition is in the form of a powder, tablet, capsule, gel or combination thereof.

14. The composition of claim 1, wherein the composition is an antimicrobial, sanitizer and/or disinfectant.

15. A product comprising the composition of claim 1, wherein the product is a cleaning composition, consumer product or personal care product.

16. The product of claim 15, wherein the product is a laundry detergent, laundry additive, fabric sanitizer, automatic dishwashing detergent, manual dishwashing detergent, multi-surface cleaner, hard surface cleaner, rinse-free antibacterial hard surface soap, hand soap, body soap, rinse-free hand soap, hand sanitizer, shampoo or animal cleaning product.

17. The product of claim 16, wherein the product is a sulfate free, natural, organic or certified organic product.

18. The product of claim 16, wherein the product is a sanitizer and/or disinfectant.

19. A reusable cleaner packaging system comprising:
(a) a container comprising a sustainable material;
(b) a lid; and
(c) the cleaning composition of claim 1 in concentrate in the form of a powder, tablet or gel.

20. The reusable cleaner packaging system of claim 19, wherein the reusable cleaner packaging system results in usage of at least 50% less packaging than conventional disposable packaging systems.
